(19) United States
(12) Patent Application Publication
(10) Pub. No.: US 2026/0210781 A1
(43) Pub. Date: Jul. 23, 2026

(54) FORCE SENSOR

(71) Applicant: CAMBRIDGE MECHATRONICS LIMITED, Cambridge (GB)

(72) Inventors: Andrew Benjamin Simpson Brown, Cambridge (GB); Benjamin Johnson, Cambridge (GB); Dominic Webber, Cambridge (GB)

(21) Appl. No.: 19/142,528

(22) PCT Filed: Dec. 20, 2023
(86) PCT No.: PCT/GB2023/053323

§ 371 Date: Jun. 23, 2025

(30) Foreign Application Priority Data
Dec. 22, 2022 (GB) 2219526.7

(51) Int. Cl. G01L 1/22; A61B 34/00; A61B 34/30

(52) U.S. Cl. CPC G01L 1/22; A61B 34/30; A61B 34/71; A61B 34/77

(57) ABSTRACT

A method of determining a force applied between a first part and a second part connected to the first part via a shape memory alloy, SMA, element such that force is transferred via the SMA element. The force is determined based on a resistance of the SMA element or an amount of energy applied to the SMA element in order to maintain a resistance of the SMA element at a predetermined value.

### FIELD

The present application relates to shape memory alloy force sensors and methods of measuring forces using the same. The present application also relates to tools including shape memory alloy force sensors.

### BACKGROUND

Force sensors are used to facilitate interactions with physical systems. Force sensors may be used to acquire quantitative data by converting an applied force into an electrical output.

A common type of force sensor is a strain gauge. Strain gauges are structured so that an electrical resistance of the strain gauge varies when a force is applied (resulting in straining). Strain gauges are often fabricated on flexible films with the resistive elements arranged in a Wheatstone bridge arrangement. Once a conversion metric is obtained through calibration, a force can be calculated based on resistance of the strain gauge. Strain gauges may be produced as reasonably thin sensors, however there are still limitations of their application due to the area of the film itself and the associated wiring to make an electrical connection. Drawbacks of strain gauges include that the structural element they are attached to needs to be significantly compliant to enable the strain to be large enough to be measured. Additionally, since the sensor is attached to a structural element, the strain gauge increases the size and may not be applicable to some geometries such as cables or wires. Furthermore, the materials used to form strain gauges are typically unsuitable for high temperatures and/or chemically sensitive environments.

Another type of force sensor is a piezoelectric force sensor. Piezoelectric sensors may be based on polymers or ceramics, and typically may provide measurable signals in response to much smaller strains. However, piezoelectric sensors provide transient signals, and are difficult to employ for accurate measurement of static or slowly varying forces. Reasonably high gain amplification is also typically needed, meaning that piezoelectric force sensors can be susceptible to electromagnetic interference.

Josephine Selvarani Ruth D and K. Dhanalakshmi, “Shape Memory Alloy Wire for Force Sensing”, IEEE SENSORS JOURNAL, VOL. 17, NO. 4, Feb. 15, 2017, describes using a shape memory alloy (SMA) wire stretched between the free end of a cantilevered beam and a structure supported the fixed beam end. The SMA wire is angled to the cantilevered beam, and is used to infer a force loading the end of the cantilevered beam.

### SUMMARY

According to a first aspect of the present invention, there is provided a method of determining a force applied between a first part and a second part. The second part is connected to the first part via a shape memory alloy, SMA, element such that force is transferred via the SMA element. The force is determined based on a resistance of the SMA element. Alternatively, the force is determined based on an amount of energy applied to the SMA element in order to maintain a resistance of the SMA element at a predetermined value.

The first and second parts may be spaced apart in a first direction. The first part may include, or take the form of, a crimp securing the SMA element. The second part may include, or take the form of, a crimp securing the SMA element. The SMA element may extend in the first direction to connect between the first and second parts. The resistance of the SMA element takes the form of the electrical resistance between the first and second parts along the SMA element.

The SMA element may include, or take the form of, a SMA wire or a length thereof. The SMA element may include, or take the form of, a cylinder. The SMA element may include, or take the form of, a rod. The SMA element may have a constant cross-section shape and/or area when moving along a direction parallel to the applied force. The SMA element may have variable cross-section shape and/or area when moving along a direction parallel to the applied force.

A majority of the force applied between the first part and the second part may be transferred via the SMA element. The SMA element may provide the only non-frictional force opposing movement of the second part relative to the first part parallel to the first direction. In this way, the SMA element may act as a force-transmitting element. One or more bearings, guides or comparable structures may provide constraint of degrees of freedom other than parallel to the first direction.

A majority of the force may correspond to 50% or more of the applied force. The SMA element may transfer 60% or more of the applied force. The SMA element may transfer 70% or more of the applied force. The SMA element may transfer 75% or more of the applied force. The SMA element may transfer 80% or more of the applied force. The SMA element may transfer 90% or more of the applied force. The SMA element may transfer 95% or more of the applied force. The SMA element may transfer all, or substantially all, of the applied force.

Determining a force may comprise determining a magnitude of the force.

The force may be applied along the SMA element parallel to a first direction. The SMA element may be configured such that the applied force corresponding to the upper plateau stress of the material of the SMA element varies with position along the first direction.

The configuration of the SMA element such that the applied force corresponding to the upper plateau stress of the SMA material varies with position along first direction may correspond to the physical shape of SMA element. Additionally or alternatively, the configuration of the SMA element such the applied force corresponding to the upper plateau stress of the SMA material varies with position along first direction may correspond to the shape and/or arrangement of one or more structural elements within which the SMA element is embedded or to which the SMA element is attached.

The SMA element may be configured such that the applied force generates a stress gradient along the SMA element. The stress gradient may be between the first and second parts. The stress gradient may be along the first direction.

A temperature gradient may be applied or generated along the SMA element. The temperature gradient may be between the first and second parts. The temperature gradient may be along the first direction. The temperature gradient may be generated by resistive heating of the SMA element using a drive current.

A cross-sectional area of the SMA element may taper along the length of the SMA element. The SMA element may taper between the first and second parts. The SMA element may taper in the first direction.

At least part of the length of the SMA element may be embedded in, or bonded to, a second material. The spring constant of the second material may taper along the length of the SMA element.

The spring constant of the second material corresponds to the constant of proportionality between applied force transferred via a length of the second material and extension of that length of the second material. The spring constant of the second material may taper along the length of the SMA element by tapering the cross-sectional area of the second material. The spring constant of the second material may taper along the length of the SMA element by changing stiffness of the second materials. For example, the second material may be a blend/composite of two materials having varying volume fractions (e.g. matrix and reinforcing fibres), or the second material may have varying volume fraction or porosity. The spring constant of the second material may taper along the length of the SMA element by varying an shape of the second material. For example, depth and or spacing of crenellations (or similar) in an exterior surface of the second material may be varied.

The second material may taper in the first direction. Substantially all of the length of the SMA element may be embedded in, or bonded to, the second material. At least 95% of the length of the SMA element may be embedded in, or bonded to, the second material. At least 90% of the length of the SMA element may be embedded in, or bonded to, the second material. At least 85% of the length of the SMA element may be embedded in, or bonded to, the second material. At least 80% of the length of the SMA element may be embedded in, or bonded to, the second material. At least 75% of the length of the SMA element may be embedded in, or bonded to, the second material.

The second material may include, or take the form of, a sheath surrounding and bonded to the SMA element. The second material is preferably more compliant than the SMA element. The second material may include, or take the form of, a compliant material such as an elastomeric material.

The SMA element may be directly connected to the first part, and a portion of the length of the SMA element may be engaged with a block of compliant material which couples the SMA element to the second part.

The portion of the length of the SMA element may be embedded in, or bonded to, the block of compliant material. The portion of the length of the SMA element may be at least 50%. The portion of the length of the SMA element may be at least 75%. The portion of the length of the SMA element may be at least 80%. The portion of the length of the SMA element may be at least 90%. The portion of the length of the SMA element may be at least 95%. The second part may include or more extensions which overlap the SMA element along the first direction. The block of compliant material may be received within the second part.

Determining the force based on an amount of energy applied to the SMA element in order to maintain a resistance of the SMA element at a predetermined value may include controlling a power applied to cause resistive heating of the SMA element in order to maintain the length of the SMA element at a constant value, and determining the applied force based on the power applied.

The length of the SMA element may be maintained at a constant value by monitoring the resistance of the SMA element, i.e. by controlling the power applied to maintain the resistance of the SMA element at a constant value.

The method may also include an initial step of controlling a power applied to cause resistive heating of the SMA element such that a power history enables determining the position of the SMA element in a hysteresis behaviour of the SMA material. For example, during the initial step the SMA element may be heated to a temperature sufficient to cause all, or substantially or, of the martensitic phase to be converted to the austenitic phase, followed by allowing the SMA element to cool to an initial operating value of applied power.

According to a second aspect of the invention, there is provided a force sensor including a first part and a second part. The second part is connected to the first part via a shape memory alloy, SMA, element such that in response to a force applied between the first and second parts, a force is transferred via the SMA element. The force sensor is configured to determine the force applied between the first and second parts based on a resistance of the SMA element. Alternatively, the force sensor is configured to determine the force applied between the first and second parts based on an amount of energy applied to the SMA element in order to maintain a resistance of the SMA element at a predetermined value.

The force sensor may be for use in the method of the first aspect. The force sensor may include features corresponding to any features of the method of the first aspect. Any definitions applicable to the method of the first aspect (or features thereof), may be equally applicable to the force sensor (or corresponding features thereof).

The force sensor may be configured to measure a resistance of the SMA element by further including electrical connections arranged to measure the resistance of the SMA element between the first and second parts. Additionally and optionally, the force sensor may include control circuitry configured for measurement of the resistance of the SMA element. The force sensor may be configured for measuring a resistance of the SMA element by further including a bridge circuit including the SMA element.

The force sensor may be configured such that a majority of the force applied between a first part and a second part is transferred via the SMA element.

The force sensor may take the form of a load cell.

The force may be applied along the SMA element parallel to a first direction. The SMA element may be configured such that the minimum applied force necessary for superelastic deformation of the SMA element varies with position along the first direction.

The SMA element may be configured such that the applied force generates a stress gradient along the SMA element.

The force sensor may be configured to apply or generate a temperature gradient along the SMA element.

A cross-sectional area of the SMA element may taper along the length of the SMA element.

At least part of the length of the SMA element may be embedded in, or bonded to, a second material. The spring constant of the second material may taper along the length of the SMA element.

The SMA element may be directly connected to the first part. A portion of the length of the SMA element may be engaged with a block of compliant material which couples the SMA element to the second part.

According to a third aspect of the invention, there is provided a controller configured to determine a force applied between a first part and a second part of a force sensor according to the second aspect. The force is determined based on a resistance of the SMA element. Alternatively, the force is determined based on an amount of energy applied to the SMA element in order to maintain a resistance of the SMA element at a predetermined value.

The controller may be configured to implement the method of the first aspect. The controller may include features corresponding to any features of the method of the first aspect and/or the force sensor of the second aspect. Any definitions applicable to the method of the first aspect (or features thereof) and/or the force sensor of the second aspect (or features) may be equally applicable to the controller (or corresponding features thereof).

The controller may be further configured to supply power to cause resistive heating of the SMA element.

The controller may be further configured to control the power applied to cause resistive heating of the SMA element in order to maintain the length of the SMA element at a constant value, and to determine the applied force based on the power applied.

According to a fourth aspect of the invention, there is provided a computer program stored on a non-transitory computer readable medium. When executed by a digital electronic processor, the computer program causes the digital electronic processor to carry out the method according to the first aspect.

The computer program may include features corresponding to any features of the method of the first aspect, the force sensor of the second aspect and/or the controller of the third aspect. Any definitions applicable to the method of the first aspect (or features thereof), the force sensor of the second aspect (or features) and/or the controller of the third aspect (or features thereof) may be equally applicable to the computer program (or corresponding features thereof).

Apparatus may include the force sensor according to the first aspect and the controller according to the third aspect, connected to the force sensor.

According to a fifth aspect of the invention, there is provided a tool including an actuator mechanically coupled to an operating element by a force transmission mechanism. The force transmission mechanism includes a first SMA element configured to transmit force between the actuator and the operating element.

The tool may include features corresponding to any features of the method of the first aspect, the load cell of the second aspect, the controller of the third aspect and/or the computer program of the fourth aspect. Any definitions applicable to the method of the first aspect and/or features thereof; the load cell of the second aspect and/or features thereof; the controller of the third aspect and/or features thereof; and/or the computer program of the fourth aspect and/or features thereof, may be equally applicable to the tool.

The first SMA element may include, or take the form of, a SMA wire or a length thereof. The first SMA element may include, or take the form of, a cylinder. The first SMA element may include, or take the form of, a rod. The first SMA element may have a constant cross-section shape and/or area when moving along a direction parallel to the applied force. The first SMA element may have variable cross-section shape and/or area when moving along a direction parallel to the transmitted force.

The force transmission mechanism may include one or more elements selected from linkages, machines, cables, chains, belts, pulleys, wheels, hydraulic elements, pneumatic elements, pivots, joints, gears and so forth.

The actuator may be manually driven. The actuator may include, or take the form of, an input for manual force such as, for example, a lever, a wheel, a cable and so forth. The actuator may be electrically driven. The actuator may be hydraulically driven. The actuator may be pneumatically driven. The actuator may include, or take the form of, a motor, a generator, a valve, a shape memory alloy actuator, and so forth.

The first SMA element may be the ultimate or penultimate element of the force transmission mechanism.

The first SMA element may be configured to undergo superelastic deformation in response to a predetermined threshold transmitted force. In this way, the first SMA element may be used to prevent the operating element outputting excessive force. In other words, the first SMA element may function as “physical fuse”.

The predetermined threshold transmitted force is predetermined in that it is set in advance of obtaining one of more measurements. The predetermined threshold transmitted force may be set by controlling the shape and/or material of the first SMA element.

The tool may be configured to apply resistive heating to the first SMA element to control a temperature of the SMA element in order to reset the first SMA element to an original shape following superelastic deformation, and/or to vary the temperature of the first SMA element in order to adjust the predetermined threshold transmitted force.

The tool may be further configured to determine the force transmitted by the first SMA element.

The force transmitted by the first SMA element may be determined using the method according to the first aspect.

The tool may be further configured to obtain or measure a force output by the actuator, to measure the force transmitted by the first SMA element, and in response to a difference between the force output by the actuator and the force transmitted by the first SMA element, to control the temperature of the first SMA element to compensate for the difference.

Measurement of the force transmitted by the SMA element is preferably, though not essentially, according to the method of the first aspect.

The force output by the actuator may be obtained or determined using any suitable force sensor such as, without limitation, a strain gauge, a piezoelectric sensor, the method of the first aspect and/or the force sensor of the second aspect, and so forth.

The temperature control of the first SMA element may be provided by driving a current through the first SMA element to cause Joule heating. The temperature control of the first SMA element may be provided by a separate heating element in thermal contact with the first SMA element.

The tool may be further operable in a stabilised mode in which the tool is configured to measure the force transmitted by the first SMA element, and in response to a difference between the force transmitted by the first SMA element and a target force, to control the temperature of the first SMA element to compensate for the difference.

The target force may be predetermined, user determined or automatically determined. The target force may be determined based on a moving window average of recently measured force values. For example, a user may adjust the tool to a desired force, then actuate a switch or toggle element provided on the tool to activate the stabilised mode to lock the transmitted force to the target force. This may be particularly advantageous when the actuator is manually driven.

When the actuator is not manually driven, the target force may correspond to the intended actuator output force. For example, in order to set an intended target force, the tool may look-up a corresponding drive current for a motor etc. The target force may then be used to perform corrections for any fluctuations in the transmitted force using the first SMA element.

The tool according may be further operable in a stabilised mode in which the tool is configured to determine a length of the first SMA element based on the resistance of the first SMA element, and in response to a difference between the length of the first SMA element and a target length, to control the temperature of the first SMA element to compensate for the difference.

The target length may be predetermined, user determined or automatically determined. The target length may be determined based on a moving window average of recently measured length values. For example, a user may adjust the tool to a desired position, then actuate a switch or toggle element provided on the tool to activate the stabilised mode to lock the corresponding length of the first SMA element to the target length. This may be particularly advantageous when the actuator is manually driven.

When the actuator is not manually driven, the target length may correspond to the intended position and state of the operating element. For example, in order to set an intended target length, the tool may look-up a corresponding drive current for a motor etc. The target length may then be used to perform corrections for any fluctuations in the positioning provided using the first SMA element.

The force transmission mechanism may further include a second SMA element separated from the first SMA element by at least one other element of the transmission mechanism. The second SMA element may be connected in series with the first SMA element.

Any functions and/or features described in relation to the SMA element may be additionally or alternatively implemented using the second SMA element.

The tool may be further configured to cyclically heat the first SMA element, and optionally the second SMA element, to cause vibrations of the force transmission mechanism.

The generated vibrations of the force transmission mechanism are preferably of low amplitude and/or high frequency. Closed loop control is preferably implemented to ensure the contraction of the first SMA element is equal to the extension of the other, opposing second SMA element. The frequency of vibrations may be greater than or equal to 10 Hz. The frequency of vibrations may be greater than or equal to 20 Hz. The frequency of vibrations may be greater than or equal to 50 Hz.

The generated vibrations of the force transmission mechanism may reduce sticking in the force transmission mechanism arising from one or more elements transitioning between static and dynamic friction. When two SMA elements are present and connected in series with each other, the first and second SMA elements may be controlled such that contraction of the first SMA element causes expansion of the second SMA element and vice versa.

The operating element may be configured for gripping. The operating element may be configured for cutting. The tool may be a surgical tool. The surgical tool may be for keyhole surgery.

The force transmission mechanism may include, or take the form of, one or more cables. The first SMA element may be connected in series with at least one cable of the one or more cables.

The force transmission mechanism may include, or take the form of, a cable-pulley drive. The first SMA element may be connected in series with at least one cable of the cable-pulley drive.

When included, the second SMA element may be disposed at the opposite end of the cable-pulley drive to the first SMA element. For example, the first SMA element may connect a first end of the cable-pulley drive to the operating element, whilst the second SMA element connects a second end of the cable-pulley drive to the actuator.

The actuator may include, or take the form of, a servo-actuator.

### BRIEF DESCRIPTION OF THE DRAWINGS

Certain embodiments of the present invention will now be described, by way of example only, with reference to the accompanying drawings, in which:

FIG. 1 is a schematic block diagram of a force sensor;

FIG. 2 is a schematic cross-section of a first exemplary force sensor;

FIG. 3 is a schematic cross-section of a second exemplary force sensor;

FIG. 4 is a schematic stress-strain curve for a shape memory alloy material;

FIG. 5A is a schematic cross section of a third exemplary force sensor;

FIG. 5B illustrates a stress gradient generated in the third exemplary force sensor shown in FIG. 5A;

FIG. 6 is a schematic cross section of a fourth exemplary force sensor;

FIG. 7 is a schematic cross section of a fifth exemplary force sensor;

FIG. 8A illustrates the effect of increasing temperature on a stress-strain curve of a shape memory alloy material;

FIG. 8B illustrates the effect of decreasing temperature on a stress-strain curve of a shape memory alloy material;

FIG. 9A is a schematic resistance-strain profile for a shape memory alloy element;

FIG. 9B is a schematic plateau stress-temperature profile for a shape memory alloy material;

FIG. 10 is a process-flow diagram of a method of active force measurement;

FIG. 11 is a block schematic of a tool including a shape memory alloy element;

FIG. 12 schematically illustrates a first exemplary surgical tool;

FIGS. 13A and 13B schematically illustrate a force correction method; and

FIG. 14 schematically illustrates a second exemplary surgical tool.

### DETAILED DESCRIPTION

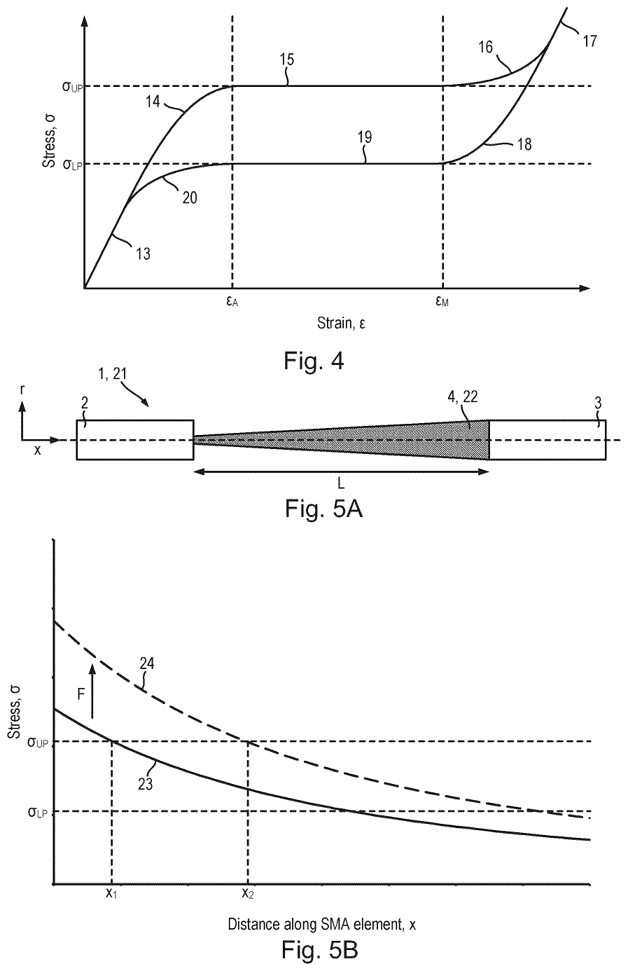

In the present specification, methods and apparatus shall be described which enable using a shape memory alloy (SMA) element as both a structural or force transmitting element, and also to measure an applied force. As described further in relation to FIG. 4, use of SMA elements to infer an applied force is difficult because SMA elements may undergo significant increases in strain with no, or minimal, changes in stress (so called “superelastic” behaviour). However, the inventors of the present specification have overcome this difficulty and devised approaches to enable correlating the resistance of an SMA element with the force applied to it.

In a first general approach, the SMA element is arranged to have a thermal or a stress gradient along its length, so that increasing stress causes progressive straining of the SMA element. The straining of the SMA element is measured by monitoring the resistance of the SMA element, which may be related to the SMA element length.

In a second general approach (in which no thermal or stress gradient along the length of the SMA element is required), power is applied to the SMA element to cause Joule heating and thereby to actively control the SMA element to a desired length (resistance) by controlling the relative fractions of austenitic and martensitic phases. The power required to achieve this desired length (correlating to a temperature of the SMA element) is used to calculate the force applied to the SMA element.

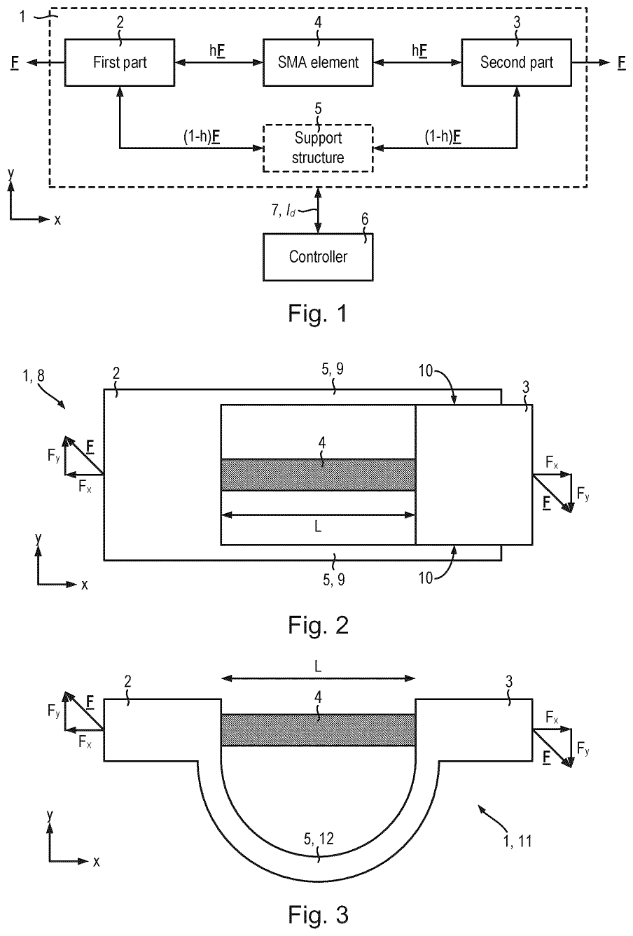

Referring to FIG. 1, a schematic block diagram of a force sensor 1 is shown.

The force sensor 1 includes a first part 2 and a second part 3. The first 2 and second 3 parts are connected together by a SMA element 4 such that force F applied between the first 2 and second 3 parts is transferred at least partly via the SMA element 4. Optionally, the first 2 and second 3 parts may also be connected by a support structure 5. A fraction hF of the force F is transferred via the SMA element 4 and, if a support structure is present, the remainder (1−h)F is transferred via the support structure 5 (h=1 when there is no support structure). In general, if the force F is not aligned uniaxially with the force sensor 1, then the fraction of each component transferred via the SMA element 4 may vary, i.e. for force F=(Fₓ, F_{y}, F_{z}), the SMA element 4 may transfer a fraction hₓFₓ along the first direction x, a fraction h_{y}F_{y} along a second direction (y-direction) and a fraction h_{z}F_{z} along a third axis (z-direction).

The first 2 and second 3 parts are spaced apart in a first direction (x-axis, as illustrated). The SMA element 4 extends in the first direction x to connect between the first 2 and second 3 parts. The resistance R of the SMA element 2 takes the form of the electrical resistance between the first 2 and second 3 parts along a length L of the SMA element 4. The SMA element 2 may be formed from nickel titanium (nitinol), copper-aluminium-nickel, or any other alloy demonstrating shape-memory alloy effects.

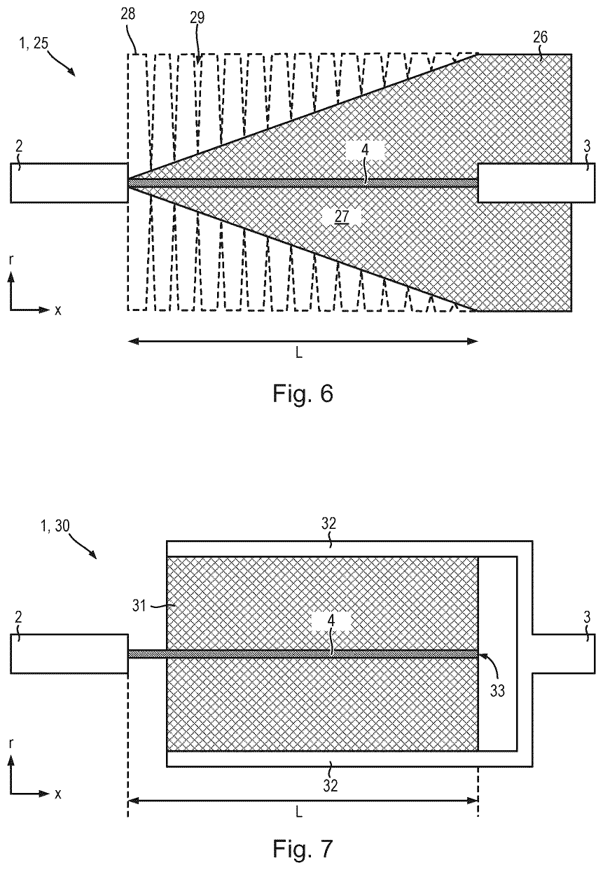

The SMA element 4 is not particularly limited in size or shape, provided that the geometric dimensions of the SMA element 4 have been characterised. Examples of the SMA element 4 may include, without limitation, an SMA wire or a length thereof, a cylinder, a rod and so forth. In some examples, the SMA element 4 may have a constant cross-section shape and/or area along the first direction x (see for example FIGS. 6 and 7). Alternatively, in other examples the SMA element 4 may have variable cross-section shape and/or area along the first direction x (see for example FIGS. 5A and 5B).

The first 2 and second 3 parts include fastening/connecting means for installation and reliable transfer of force F to the SMA element 4. For example, is the SMA element 4 is a wire or rod, then the first 2 and second 3 parts may each include, or take the form of, crimp heads securing the SMA element 4.

The force F is determined based on a resistance R of the SMA element 4 of the force sensor 1. As illustrated in FIG. 1, a controller 6 is coupled to the force sensor 1 by an electrical coupling 7 (for example, two or more wires). The controller 6 is configured to measure the resistance of the SMA element 4, and based on this to infer a strain ε of the SMA element 4 along the length of the SMA element 4 (along the first direction x). Based on the strain ε, the fraction hₓFₓ of applied force F along the length of the SMA element 4 may be determined, and from appropriate pre-calibrations the applied force F. Although any suitable resistance R measurement technique may be used, a Wheatstone bridge resistance measurement is preferable. The force sensor 1 may include only connections for coupling 7 to the controller 6, or alternatively other components of a Wheatstone bridge and/or other control/measurement circuitry may also be integrated as part of the force sensor 1. In some examples, the controller 6 may be integrated as part of a single package with the force sensor 1.

In addition to resistance measurements, the controller 6 may also use the electrical coupling 7 to drive current I_{d} through the SMA element 4 to heat the SMA element 4 to within the transition zone between martensitic and austentic phases with Joule heating I_{d}²R. As described hereinafter, in some examples, the precise quantity of power I_{d}²R delivered may be used as part of the method for determining the applied force F.

An example of a resistance feedback control technique which may be implemented using the controller 6 is described in WO 2014/076463 A1, which is incorporated herein by this reference. Useful background for methods of driving SMA wires 19, 22 is also provided in WO 2013/175197 A1 and also WO 2019/073212 A1, both of which are incorporated herein by this reference.

### First Exemplary Force Sensor

Referring also to FIG. 2, a schematic cross-section of a first exemplary force sensor 8 is shown (hereinafter the “first sensor”).

In the first sensor 8, a majority of the force F applied between the first part 2 and the second part 3 is transferred via the SMA element 4. In other words, the fraction h>0.5, and preferably h>>0.5, for example h>0.95.

In the first sensor 8, the support structure 5 takes the form of a hollow, prismatic extension 9 extending along the first direction x from the first part 2. For example, if the first part 2 is circular in the y-z plane, the extension 9 will take the form of a hollow cylinder and so forth. The second part 3 is received within the extension 9, such that degrees of freedom of the second part 3 relative to the first part 2 are constrained except along the first direction x. In this way, the SMA element 4 coupled between the first 2 and second 3 parts provides the only non-frictional force opposing movement of the second part 3 relative to the first part 2 parallel to the first direction x. In other words, the SMA element 4 transmits substantially all of the component Fₓ of an applied force F along the first direction x. Preferably, the support structure 5 bears substantially all of any perpendicular component F_{y}. For example, the hollow extension 9 may be straightforwardly configured for significantly greater flexural rigidity than the SMA element 4.

In FIG. 2, the first sensor 8 is illustrated with the second part 3 and the extension 9 in sliding contact at an interface 10. However, one or more bearings may be disposed at the interface to reduce friction between the extension 9 and second part 3.

### Second Exemplary Force Sensor

Referring also to FIG. 3, a schematic cross-section of a second exemplary force sensor 11 is shown (hereinafter the “second sensor”).

The first sensor 8 is simple to interpret because the component of force along the first direction is essentially all transferred via the SMA element 4. This configuration may not be ideal for all applications. For example, when the magnitude F of the force F is larger, the size of SMA element 4 needed to support the entire load may become prohibitively large and consequently difficult to heat with Joule heating and/or slow to respond to changes in temperature due to increased thermal mass. Additionally, unless controlled using one of the active configurations described herein, the first sensor 8 will exhibit poor linearity of the SMA response due to elongating at a substantially constant stress (see σᵤₚ in FIG. 4). This means that the length L and resistance R of the SMA element 4 will change suddenly at this stress σᵤₚ whereas for a force sensor it is desirable that the resistance changes in proportion to the force applied. The configurations described herein represent a variety of approaches to causing the stress induced transition in an SMA element 4 to occur over a larger range of stresses σ.

The second force sensor 11 includes a support structure 5 in the form of a flexure 12 connecting the second 2 and third 3 parts, mechanically in parallel with the SMA element 4. The material, shape and dimensions of the flexure 12 may be controlled using conventional mechanical design processes to provide the desired combination of load bearing capacity, compliance parallel to the first direction x and compliance in directions perpendicular to the first direction x.

### Stress-Strain Characteristics of Shape Memory Alloys

Presuming axial orientation, force F=|F| experienced by an object (of constant cross-section) is a product of the stress, σ, and the cross-sectional area, A:

$$F = σ A \qquad ( 1 )$$

and for regular elastic materials the stress σ is related to strain ε via the Young Modulus E:

$$σ = ε E \qquad ( 2 )$$

so that:

$$F = ε EA \qquad ( 3 )$$

However, whilst it is relatively straightforward to determine the length L of an SMA element 4 (and hence strain ε) from variations in measured resistance R, Equations (1) to (3) cannot be used to recover the magnitude F of an applied force F because SMA materials do not exhibit linear elastic behaviour above a threshold stress level.

Referring also to FIG. 4, a schematic stress-strain, Q-E, curve for a SMA material is shown.

In an initial, linear-elastic region 13, strain ε increases linearly with stress σ. As stress σ increases through a transitional region 14, the relationship to strain ε departs from linear elasticity as some regions of the austenitic phase begin to undergo diffusionless (shear) transition to the martensitic phase, eventually reaching an upper plateau region 15 through which the stress σ is substantially constant at an upper plateau stress σᵤₚ and strain ε is accommodated by phase transformation of the SMA material from the austenitic to the martensitic phase. As strain ε increases further, a further transition region 16 is reached as all available austenitic phase has been converted, leading into a second, upper linear elastic region 17. If stress σ is increased further, exceeding an ultimate strength (not shown), permanent plastic deformation may result. Plastic deformation would represent unintended and unwanted behaviour in a force sensor, and is unless otherwise specified not relevant to the presently disclosed force sensors and tools using the same.

When unloading, the upper linear elastic region 17 continues down to a stress σ below the upper plateau stress σᵤₚ before entering a transition region 18 as martensitic phase begins to undergo diffusionless (shear) transformation back to the austenitic phase. A lower plateau region 19 follows, through which strain is reduced by phase transformation of the SMA material from the martensitic to the austenitic phase at a substantially constant lower plateau stress σ_{LP}. As the transformation to austenitic phase is completed, the curve transitions 20 back to re-join the lower linear elastic region 13.

This stress-strain behaviour of shape memory alloys is sometimes termed “superelasticity”, and as shown in FIG. 4 illustrates significant hysteresis. Additionally, the stress-strain curve is a function of temperature of the SMA material, with increasing temperature shifting the plateau stresses σᵤₚ, σ_{LP} higher and decreasing temperature shifting the plateau stresses σᵤₚ, σ_{LP} lower.

For this reason, whilst length L changes 6L of the SMA element 4 are detectable by measuring the resistance R, simple conversion back to a corresponding stress σ (and hence force F) is not possible because:
There is no one-to-one mapping on the stress-strain curve; and
A single plateau stress σᵤₚ, σ_{LP} may correspond to a wide range of strains ε.

The inventors of the present specification have devised two broad approaches to overcoming this fundamental issue:
A. A passive approach wherein the force sensor 1 is configured such that the applied force F corresponding to the plateau stress (either upper σᵤₚ or lower σ_{Lp}) varies with position along the length of the SMA element 4; and/or
B. An active approach, based on controlling the power supplied to the SMA element 4 for Joule heating in order to control the length of the SMA element 4 (as determined by resistance R measurements) to a fixed value.

The passive and active approaches to providing an SMA based force sensor 1 are described in detail hereinafter.

### Passive Configurations

The first approach is to configure the SMA element 4 of the force sensor 1 such that the applied force F corresponding to the plateau stress (either upper σᵤₚ or lower σ_{Lp}) varies with position along the length of the SMA element 4.

This may be achieved in practice by configuring the SMA element 4 to experience a stress and/or temperature gradient along the length L of the SMA element 4 (i.e. between the first 2 and second 3 parts). In general this is possible by controlling one or more of a physical shape of the SMA element 4 and/or the shape and arrangement of one or more structural elements within which the SMA element 4 is embedded or to which the SMA element 4 is attached, bonded or otherwise mechanically coupled. A temperature gradient may be generated by resistive heating of the SMA element 4 using a drive current, or by auxiliary heat sources. For example separate resistive heating coils wrapped around or received/embedded within the SMA element 4.

### Third Exemplary Force Sensor

Referring also to FIG. 5A, a schematic cross section of a third exemplary force sensor 21 (hereinafter “third sensor”) is shown.

The third sensor 21 is an example of generating a stress gradient in the SMA element 4, and includes a tapering SMA element 22 coupling the first 2 and second parts 3. The third sensor 21 may include any type of support structure 5 described herein, but this is omitted from FIG. 5A for visual clarity. The SMA element 4 is cylindrically symmetric about the first direction x, and a cross-sectional area of the SMA element 4 tapers along the length of the SMA element 4. Specifically, the radius of the SMA element 4 increases linearly along the length l. The taper is illustrated in FIG. 5A from the second part 3 towards the first part 2, but equally the cross-sectional area of the SMA element 4 could taper in the opposite direction.

With the component Fₓ acting along the length L of the SMA element (parallel to the x-axis as illustrated), the stress at a given points is σ=Fₓ/A, such that with varying radius r(x):

$$σ ( x ) ∝ \frac{{F}_{x}}{{r ( x )}^{2}} \qquad ( 4 )$$

Referring also to FIG. 5B, this dependence of stress σ with position is shown for a pair of forces and for linearly varying radius r as illustrated in FIG. 5A.

A first stress gradient 23 corresponds to a first force F¹ₓ, for which the upper plateau stress σᵤₚ occurs at a first location x₁ along the SMA element 4. In this way, the regions of the SMA element 4 to either side of a narrow region will be within the linear elastic regions 13, 17 of the stress-strain curve (see FIG. 4). If the force is increased to F²ₓ>F¹ₓ, then a second stress gradient 24 is experienced, and the location experiencing the upper plateau stress σᵤₚ is shifted to a second location x₂.

In this way, whilst the stress-strain curve of each local volume of SMA material still exhibits plateaus σᵤₚ, σ_{LP}, the overall force-strain curve (force vs engineering strain ΔL/L of the SMA element 4 overall) does not, allowing strain ε of the SMA element (determined from the resistance R) to be correlated to an applied component of force Fₓ along the SMA element 4. In use, the thermomechanical history of the SMA element 4 should be tracked so that it can be determined which branch of the stress-strain hysteresis curve the SMA material is experiencing (within the localised region experiencing the plateau stress σᵤₚ or σ_{LP} at a given applied force).

Although explained in relation to tapering of a cylindrically symmetric SMA element 4 such as a wire, any shape having a cross-sectional area A varying with position x along the first direction may be used. For example, the SMA element 4 may be wedged shaped, such as a film or foil of varying thickness.

Preferably, the SMA element 4 is also heated, for example by Joule heating using a drive current I_{d}, and the tapering area A will also vary the resistance and hence localised resistive heating I_{d}²R, and the rate of heat radiated and hence lost, leading to a gradient in temperature as well as stress σ.

### Fourth Exemplary Force Sensor

Referring also to FIG. 6, a schematic cross section of a fourth exemplary force sensor 25 (hereinafter “fourth sensor”) is shown.

The fourth sensor 25 is an example of generating a stress gradient in the SMA element 4, and includes a SMA element 4 of constant cross-section embedded in and bonded to a cylindrically symmetric second material 26 which includes a tapering region 27 which corresponds to the length L of the SMA element 4 between the first 2 and second 3 parts. The fraction of the force component Fₓ acting between the first 2 and second 3 parts which is supported by the SMA element varies, increasing as the radius of the tapering region 27 decreases. In this way, the overall force-strain curve (engineering strain of the SMA element 4 overall) is modified to remove plateaus (though these of course remain an intrinsic element the stress-strain curve of the actual SMA material at a local level).

Preferably, as shown in FIG. 6, the second material 26 is also securely bonded to the second part 3. The second material 26 is preferably more compliant (less stiff) than the material of the SMA element 4. For example, the second material may include, or take the form of, a compliant material such as an elastomeric material. Preferably, as shown in FIG. 6, the entire span of the SMA element 4 between the first 2 and second 3 parts is embedded (or otherwise engaged with) the second material 26. However, in some examples a smaller fraction of the length L of the SMA element 4 may be embedded/engaged with the second material 26.

It is important that the interfacial adhesion between the SMA element 4 and the second material 26 is strong, such that there is no delamination/slippage between the two.

Preferably, the SMA element 4 is also heated, for example by Joule heating using a drive current I_{d}, and the tapering region 27 of the second material 26 will also vary a flux of heat away from the SMA element 4 leading to a gradient in temperature as well as stress σ.

Although shown as tapering from the second part 3 towards the first part 2 in FIG. 6, equally the tapering region 27 could taper from the first part 2 towards the second part 3. Additionally, the second material 26 need not be cylindrically symmetric, for example, if the SMA element 4 takes the form of a film or plate, then the tapering region 27 may take the form of a wedge.

Furthermore, the general principle of the fourth sensor 25 is not limited to generating a stress gradient by including a tapering section 27. Any approach resulting in an overall spring constant variation of the second material 26 along the length L of the SMA element 4 may be employed. Herein, the spring constant of the second material 26 means the constant of proportionality between applied force transferred via a length of the second material and extension of that length of the second material 26.

For example, referring again to FIG. 6, an alternative profile 28 of the second material 26 is shown. The alternative profile 28 includes cuts 29 (alternatively crenellations or other cut-outs) which become progressively shallower along the length L of the SMA element 4. The free surfaces of cuts must necessarily have zero stress, meaning that the effective load-bearing area of the second material 26 is substantially the same as using the tapering region 27.

This is just one example of how to implement a spatially varying spring constant for the second material 26. Further options include using a second material 26 which is a blend/composite of two materials having varying volume fractions (e.g. matrix and reinforcing fibres) along the length L of the SMA element 4. Alternatively, the second material may have varying volume fraction of porosity along the length L of the SMA element 4.

Although illustrated and described with a constant cross-section area A of the SMA element 4, this is not essential and the SMA element 4 may also have a varying cross-sectional area A so as to augment the stress gradient.

### Fifth Exemplary Force Sensor

Referring also to FIG. 7, a schematic cross section of a fifth exemplary force sensor 30 (hereinafter “fifth sensor”) is shown.

The fifth sensor 30 is an example of generating a stress gradient in the SMA element 4, and includes an SMA element 4 which is directly connected to the first part 2, and a portion of the length of the SMA element 4 is engaged with (embedded in, or otherwise bonded to) a block 31 of compliant material which couples the SMA element 4 to the second part 3. The portion of the length L of the SMA element 4 which is engaged with the block 31 may be at least 50%, but it preferably larger, and ideally the entire length L (the block 31 may or may not be attached to the first part 2). Compliant material herein means more compliant (less stiff) than the SMA element 4, for example an elastomeric material such as a silicone rubber.

The second part 3 includes a hollow-cylindrical extension 32 which overlaps the SMA element 4 along the first direction x towards the first part 2. The block 31 of compliant material is received within, and bonded to, the hollow-cylindrical extension 32 of the second part 3. Within a portion of the SMA element 4 not engaged with the block 31 of compliant material, all of the force Fₓ is supported by the SMA element 4. The fraction of the force Fₓ supported by the SMA element 4 decreases through the portion engaged with the block 31 of compliant material, necessarily reaching zero by the free end 33 of the SMA element 4. In this way, a stress σ gradient is generated in the SMA element 4, so that an overall force-strain curve (engineering strain) of the SMA element 4 does not include plateaus.

It is important that the interfacial adhesion between the SMA element 4 and the block 31 of compliant material is strong, such that there is no delamination/slippage between the two.

The block 31 of compliant material need not be cylindrically symmetric, for example, if the SMA element 4 takes the form of a film or plate, then the block 31 of compliant material may take the form of a cuboid, and the extension of the second part 2 may be shapes to accommodate this, for example as a rectangular prism or a pair of parallel plates sandwiching the block 31 of compliant material.

### Active Configurations

As an alternative to configuring the force sensor 1 to cause a stress and/or temperature gradient in the SMA element 4 so as to eliminate plateaus from the overall force-strain characteristic of the force sensor 1 an active measurement may be conducted based on the power supplied to heat the SMA element 4.

Power P supplied to cause resistive heating I_{d}²R of the SMA element 4 will swiftly reach equilibrium with heat losses from the SMA element 4 (sum of radiative, convective and diffusive) such that a given value of power P corresponds to a temperature T. The length L of the SMA element 4 is related to the temperature T because this varies (within a range) the relative fractions of martensitic and austenitic phases.

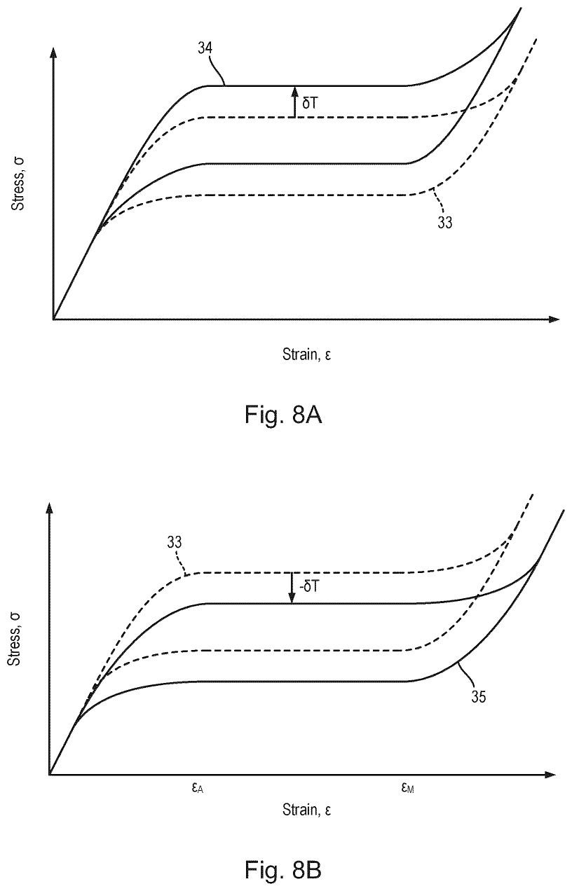

Referring also to FIG. 8A, increasing the temperature T of the SMA element 4 causes the plateaus σ_{UP} GLP to shift to higher values of stress σ.

A stress-strain curve 33 is illustrated for a temperature T and a corresponding stress-strain curve 34 is illustrated for an increased temperature T+δT.

Referring also to FIG. 8B, decreasing the temperature T of the SMA element 4 causes the plateaus σ_{UP} GLP to shift to lower values of stress σ.

A stress-strain curve 35 is illustrated for an decreased temperature T+δT, and the same stress-strain curve 33 shown in FIG. 8A for the temperature T is also shown for reference.

In the active force sensing configuration, the power P supplied to cause resistive heating I_{d}²R of the SMA element 4 is controlled in order to maintain the length L of the SMA element 4 at a fixed value L₀. The relationship between applied power P and component of force Fₓ applied to the force sensor 1 may be determined from calibration experiments for a given fixed length L₀, allowing an unknown applied force Fₓ to be inferred based on the power P.

The length L of the SMA element 4 is maintained at the fixed value L₀ by monitoring the resistance R of the SMA element 4 by the controller 6. In other words, the controller 6 varies the power P supplied so as to maintain the resistance R of the SMA element at a fixed value R₀ (corresponding to length L₀).

The resistance of an SMA element 4 of fixed cross-section is given by:

$$R = \frac{ρ L}{A} \qquad ( 5 )$$

In which ρ is resistivity, L is length of the SMA element 4 and A is the cross-sectional area. In this way, resistance R of the SMA element 4 may be calibrated to strain ε.

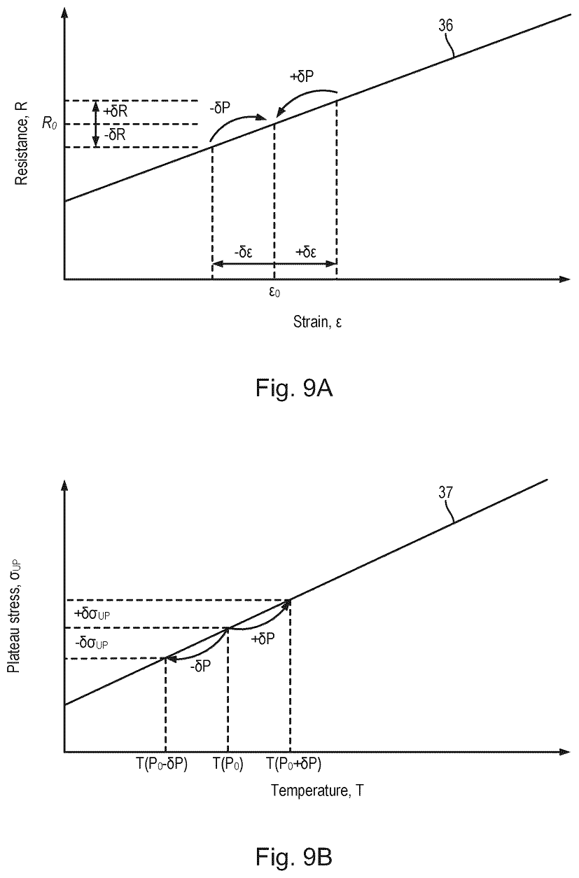

Referring also to FIG. 9A, a schematic resistance-strain profile 33 is shown.

The schematic resistance-strain profile 36 is shown as linear for ease of explanation. However, the resistance-strain profile 36 may be non-linear, and for a given force sensor 1 and SMA element 4 the resistance-strain profile 33 will need to be calibrated prior to use.

Referring also to FIG. 9B, a schematic plateau stress-temperature profile 37 is shown.

The plateau stress-temperature profile 37 corresponds to the variation of the upper plateau stress σᵤₚ as a function of temperature T (see FIGS. 8A and 8B). As discussed hereinbefore, the temperature T of the SMA element is a function of applied power P. The upper plateau stress σᵤₚ is shown, but the lower plateau stress σ_{LP} could equally be used as the behaviour with temperature is the same.

With reference in particular to FIGS. 9A and 9B, the configuration of a force sensor 1 for active strain controlled force measurements shall be described.

When the force sensor 1 is not subjected to an external force F, a baseline power P₀ is supplied (by controller 6) for Joule heating of the SMA element 4, corresponding to the fixed strain ε₀ having corresponding resistance R₀. The power P₀ corresponds to a baseline operating temperature T₀=T(P₀). The fixed strain ε₀ is not necessarily zero, since strain may be defined relative to the unloaded and un-powered SMA element (i.e. ambient temperature). The baseline operating temperature T₀ should correspond to a point where the SMA element 4 is part-way through the transition between martensitic and austentic phases.

When the controller 6 detects a change δR in the resistance R of the SMA element 4, this will correspond to a change in strain δε (see FIG. 9A).

The controller will increase or decrease the applied power P as needed to restore the strain ε (and hence resistance R) to the fixed value ε₀ (R₀). For example, if the strain ε increases (tension) by +δE, the controller 6 detects a corresponding resistance shift of δR. The controller 6 increases the power P until the resistance R returns to the fixed value R₀, corresponding to power P+δP. However, the increased power will cause the SMA element 4 to equilibrate at increased temperature T(P+δP), corresponding to an elevated plateau stress σᵤₚ. This elevated plateau stress σᵤₚ can then be converted to the applied force component Fₓ applied to the force sensor 1 using the known geometry of the SMA element 4. The same processes may be applied to an applied reduction −δR in the resistance R of the SMA element 4, by decreasing power −δP to determine an applied compressive force component −Fₓ.

In practice, because the force component Fₓ and the stress σ in the SMA element 4 are related, and because the temperature T and the power P are related, a force sensor 1 configured for active strain control may be directly calibrated to generate a curve relating force along the SMA element Fₓ to applied power P supplied by the controller 6 by applying known forces (e.g. standard masses) and measuring the power P that needs to be applied to obtain the desired operating point resistance R₀. In this way, the precise relationships of resistance R to strain ε, of power P to temperature T, or of stress σ in the SMA element 4 to force Fₓ applied between the first 2 and second 3 parts are never required to be measured.

When a force sensor 1 may be required to operate across a broader range of temperatures, significant shifts in ambient temperature T_{omb} may affect the precise relationship between supplied power P and the corresponding equilibrium temperature T(P) of the SMA element 4. This may be accounted for performing calibrations to determine applied force Fₓ vs power P characteristics at a range of ambient temperatures T_{omb} spanning a required operational range. The characteristics may be stored in the controller 6, and an ambient temperature sensor (not shown) coupled to the controller 6 to allow looking up the appropriate characteristic for the measured ambient temperature T_{omb}. When a measured ambient temperature T_{omb} is between specifically calibrated values, interpolation may be employed using techniques known from other types of sensors which employ ambient temperature corrections.

The active strain control method does require knowledge of which branch of the hysteresis loop the SMA element 4 is on, in other words, whether the SMA element 4 is operating on the upper plateau stress σ_{UP} or on the lower plateau stress σ_{LP}. This may be done by monitoring the history of the resistance R. Alternatively, when the force sensor 1 is initially switched on, the controller 6 may carry out an initial step of controlling the power P to cause resistive heating of the SMA element 4 such that the power P history enables determining the position of the SMA element 4 in the hysteresis behaviour of the SMA material. For example, during the initial step the controller 6 may cause the SMA element 4 to be heated to a temperature sufficient to cause all, or substantially all, of the martensitic phase to be converted to the austenitic phase, followed by allowing the SMA element 4 to cool to the initial operating value P₀ of applied power P.

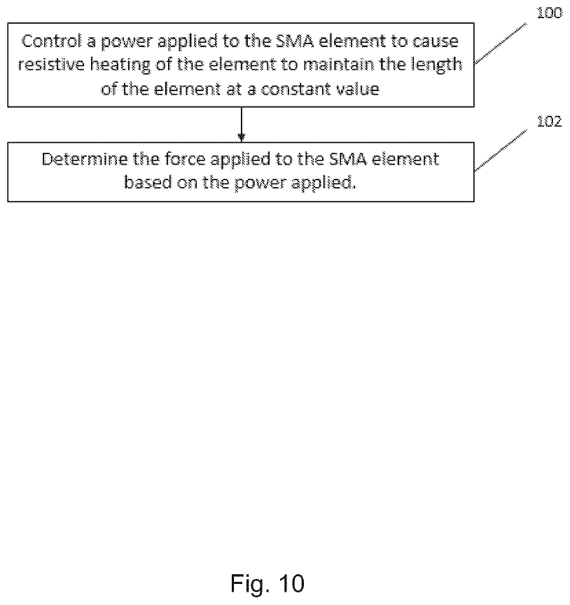

With reference to FIG. 10, a method of active force measurement is described. At step 100, power is applied to the SMA element to cause resistive heating in order to maintain the length of the element (which corresponds to a resistance of the element). At step 102, a force applied to the SMA element is determined based on the power applied. Equally, the force may be determined based on an amount of energy supplied to the SMA element.

### SMA Enabled Tool

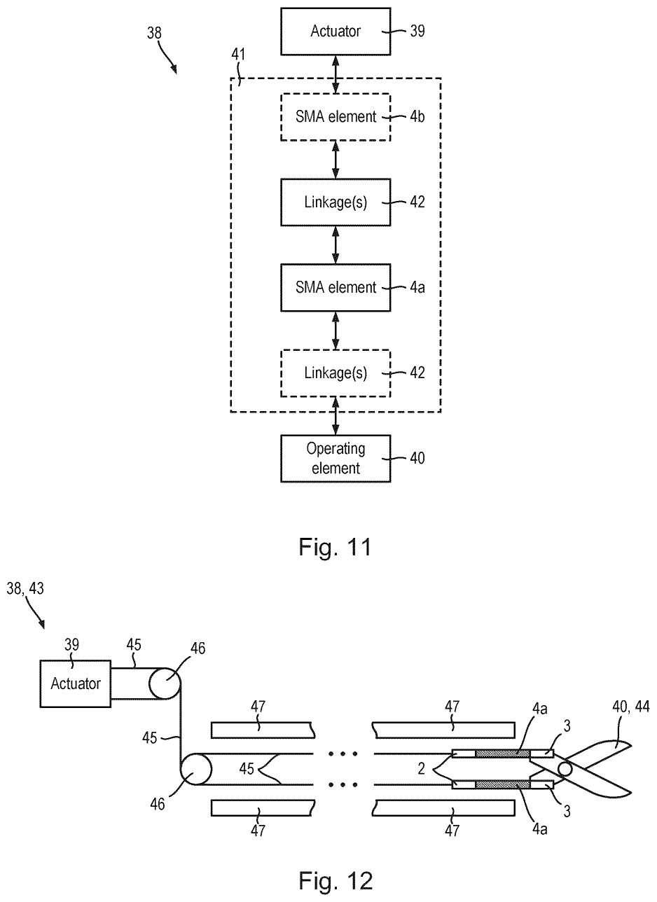

Referring also to FIG. 11, a block schematic of a tool 38 including an SMA element 4 is shown.

The tool 38 includes an actuator 39 (including manually operable mechanisms) which is mechanically coupled to an operating element 40 by a force transmission mechanism 41. The force transmission mechanism 41 includes a first SMA element 4a configured to transmit force F between the actuator 39 and the operating element 40. The force transmission mechanism 41 also includes one or more linkages 42 for transmitting the force F between the actuator 39 and the operating element 40. Although shown in FIG. 11 as being mechanically in series with the linkages 42, the first SMA element 4a may alternatively be connected in parallel across one or more of the linkages 42.

Linkages 42 may in general take any form suitable for transmitting force F, including without limitation machines, cables, chains, belts, pulleys, wheels, hydraulic elements, pneumatic elements, pivots, joints, gears and so forth.

The actuator 39 may be driven electrically, hydraulically, pneumatically or even manually. Examples of manually driven actuators 30 include, without being limited to, an input for manual force such as a lever, a wheel, a cable and so forth. Examples of non-manual actuators 39 include, without being limited to, a motor, a generator, a valve, a shape memory alloy actuator, a piezoelectric actuator, and so forth.

The first SMA element 4a is an example of the SMA element 4 described in relation to the force sensor 1, and may take any form described herein such as, for example, a wire, cylinder, rod, plate, foil and so forth. The first SMA element 4a may be part of any force sensor 1 described herein, i.e. including the first 2 and second 3 parts. Alternatively, the first SMA element 4a may form a force sensor 1 as described herein in combination with first 2 and second 3 parts which form part of adjacent (in the force transmission mechanism) linkage(s) 42 and/or the operating element 40. In some embodiments the SMA element 4 may not form part of a force sensor.

Preferably, though not essentially, the first SMA element 4a may provide (or be connected in parallel with) the ultimate or penultimate element of the force transmission mechanism 41. For example, the SMA element 4a is preferably directly coupled to the operating element 40, or is coupled to the operating element via one additional linkage 42.

The operating element 40 may be configured for gripping, cutting, slicing, or any other operation as may be required. For example, the operating element 40 may take the form of a surgical tool such as a clamp, grip, scissors, suction nozzle and so forth. The tool 38 may be particularly useful to implement surgical tools for keyhole surgery (see also FIGS. 12 to 14). The tool 38, or several tools 38, may form part of a robotic surgical system.

Alternatively, the tool 38 and operating element 40 may be configured for assembly/manufacture. For example, the operating element 40 could take the form of a suction nozzle or gripper for a pick-and-place machine, a soldering iron for soldering components, a sensor that requires contact (such as an eddy current sensor), and so forth.

The tool 38 may optionally include a second SMA element 4b in series between the force transmission mechanism 41 and the actuator 39, preferably the ultimate or penultimate element in the direction from operating element 40 to actuator 39. An example of using the second SMA element 4b are described in relation to FIG. 14.

### Excess Force Protection

The first SMA element 4a may be configured to undergo superelastic deformation in response to a predetermined threshold magnitude of transmitted force Fₜₕᵣₑₛₕ. In other words, the size, dimensions and/or materials of the first SMA element 4a are arranged so that the upper plateau stress σᵤₚ corresponds to the desired threshold force Fₜₕᵣₑₛₕ. In this way, the first SMA element 4a may be used to prevent the operating element outputting excessive force. In other words, the first SMA element may function as “physical fuse”. This may help to protect the operating element 40, and/or objects the operating element is being used on, from becoming damaged.

A single-use excess force protection may be of limited utility, in particular if the tool 38 should be re-usable. The first SMA element 4a may be heated to restore it to a trained shape, for example by immersion in hot water/oil or application of a heat gun. However, more conveniently, the tool 38 may include the controller 6 which may apply resistive heating to the first SMA element 4a to control a temperature of the first SMA element 4a in order to reset the first SMA element 4a to its original (trained) shape following an incidence of superelastic deformation. Additionally or alternatively, the controller 6 may also modify the supplied power P to vary the temperature T of the first SMA element 4a in order to adjust the threshold force Fₜₕᵣₑₛₕ. For example, the threshold force Fₜₕᵣₑₛₕ may be user adjustable (within a range).

### Force Measurements

In some examples, the tool 38 may be configured to determine the magnitude of force F transmitted by the first SMA element 4a. In such examples, the tool 38 further includes the controller 6 and the first SMA element 4a forms part of a force sensor 1. Force measurements may be conducted using the passive (stress and/or temperature gradient) configurations described hereinbefore (see FIGS. 5A to 7), the active strain configuration described hereinbefore (see FIGS. 8A to 10), or a combination of both.

The measurement of force F transferred by the first SMA element 4a may be used to provide feedback to an operator, for example using an output display. Additionally, the measurement of force F transferred by the first SMA element 4a may be incorporated into useful automatic feedback methods, examples of which are described hereinafter.

### First Exemplary Surgical Tool

Surgical tools allow specific tasks to be performed when operating. These tools can be manually operated or used in robotic surgery systems. Common between both applications is the use of cables to transmit power from the source of actuation to the distal end of the instrument where an operating element 40 performs a specific operation, such as grasping or cutting. Due to form factor constraints, force and dexterity requirements, and so forth, the cables are usually run through a series of pulleys each of which cause a loss in transmission efficiency due to the friction in pulley bearings, varying bending stiffness and fatigue of the cables. This results in positional inaccuracy in the distal mechanism. Alternatively, pulleys need not be used, and cables may simply be run through sheathes (to which the cables are not attached).

A surgical tool implemented according to the tool 38 includes the first SMA element 4a which, in addition to being used for force F measurements may also be used as an actuator itself to permit varying tension force F in the cables in order to measure and/or compensate for the losses described hereinbefore.

Referring also to FIG. 12, a first exemplary surgical tool 43 (hereinafter the “first tool”) is shown.

The first tool 43 utilizes a cable drive mechanism in series with SMA elements 4 in the form of SMA wire. The actuator 39 may be a servo actuator for robotic surgery, but, in the case of manual surgery will be the applied force of the surgeon. Force F is transmitted to the operating element 40, for example a gripper mechanism 44 as illustrated (but any other operating element 40 useful for surgery may be used). The cable drive mechanism includes a system of cables 45 and pulleys 46, and for the majority of the length between the actuator 39 and the gripper 44 the cables 45 are contained within a cable sheathe 47 which both protects and constrains the cables 45.

In the first tool 43, a pair of cables 45 are coupled to each arm of the gripper 44 via respective first SMA elements 4a. The first SMA elements 4a are placed at the distal end of the first tool 43, as the ultimate element of the force transmission mechanism 41. Each first SMA element 4a forms a force sensor 1 with a first part 2 (for example a crimp head) coupling to the respective cable 45, and the second part 3 being either attached to, or integrally formed with, one of the gripper 44 arms. In other examples, a single cable 45 opposed by a spring acting on the gripper could be used. A controller 6 (not shown in FIG. 12) is either integrated with, or coupled to, the first tool 43. Each first SMA wire 4a may be coupled to a separate controller 6, or alternatively a single controller 6 may include multiple channels, each supporting a different force sensor 1. When the first tool 43 is part of a surgical robot, the controller 6 may be an integral component of the surgical robot

Using the controller 6 and force sensors 1, force applied to (and by) the gripper 44 may be accurately and continuously monitored, enabling to surgeon using a robotic system and/or keyhole surgical tools to accurately know the applied force which an operating element 40 such as the gripper 44 exerts on the tissue being manipulated during the procedure. This may help to reduce problems of applying excess or insufficient force, either of which may potentially cause tissue damage during surgery.

When included in the first tool 43, for example to measure the force output Fₒᵤₜ by the actuator 39, a pair of second SMA elements 4b may be disposed at the opposite end of the cable-pulley drive to the first SMA elements 4a (see FIG. 14). For example, the first SMA elements 4a may connect a first end of the cable-pulley drive to the operating element, whilst the second SMA elements 4b connect a second end of the cable-pulley drive to the actuator 39.

### Force Loss Corrections

Referring also to FIG. 11, the tool 38 is not limited to only measurement of the magnitude of a force Fₜᵣₐₙₛ transmitted by the first SMA element 4a, the first SMA element 4a may also be actuated to adjust the transmitted force Fₜᵣₐₙₛ. In general, an SMA element 4, 4a 4b may be caused to contract by changing the temperature (typically on heating), or allowed to expand in response to a force (typically be allowing cooling). This may be controlled by the controller 6 using the power P supplied for Joule heating.

Actuation of the first SMA element 4a may be used to compensate for losses due to friction, mechanical compliance and so forth in linkages 42 of the force transmission mechanism 41 between the actuator 39 and the first SMA element 4A.

The force output Fₒᵤₜ by the actuator 39 is measured or otherwise obtained. For example, the force output Fₒᵤₜ may be measured using any suitable force sensor, including but not limited to a force sensor 1 as described herein incorporating the second SMA element 4b (see FIG. 14), a strain gauge, a piezoelectric force sensor, and so forth. Alternatively, when the actuator 39 has a controlled force output, the value which the actuator 39 is set to may be used as Fₒᵤₜ.

The force transmitted Fₜᵣₐₙₛ by the first SMA element 4a is measured, using any combination of force sensor 1 and method as described herein.

A difference between the force output Fₒᵤₜ and the transmitted force Fₜᵣₐₙₛ is determined, ΔF=Fₒᵤₜ−Fₜᵣₐₙₛ. If there is a difference ΔF, then the controller 6 causes actuation of the first SMA element 4a to compensate for the difference ΔF, i.e. to reduce or remove (to within accuracy of the measurement) the difference ΔF. Depending on the sign of the difference ΔF, the actuation may take the form of increasing the temperature of the first SMA element 4a (by increasing the driving current), or may take the form of allowing the temperature of the first SMA element 4a to decrease (by decreasing the driving current, coupled with natural heat losses).

Such compensations made to the transmitted force Fₜᵣₐₙₛ may help to minimize effect of mechanical compliance, backlash and so forth in the force transmission mechanism 41.

The heating of the first SMA element 4a to cause actuation has been described as originating from Joule heating by a drive current I_{d}. However, in some examples the controller 6 may control alternative heat sources which are external to the SMA element 4 such as, for example, a separate heating element in thermal contact with the first SMA element, by generation of eddy currents in the SMA element 4, and so forth.

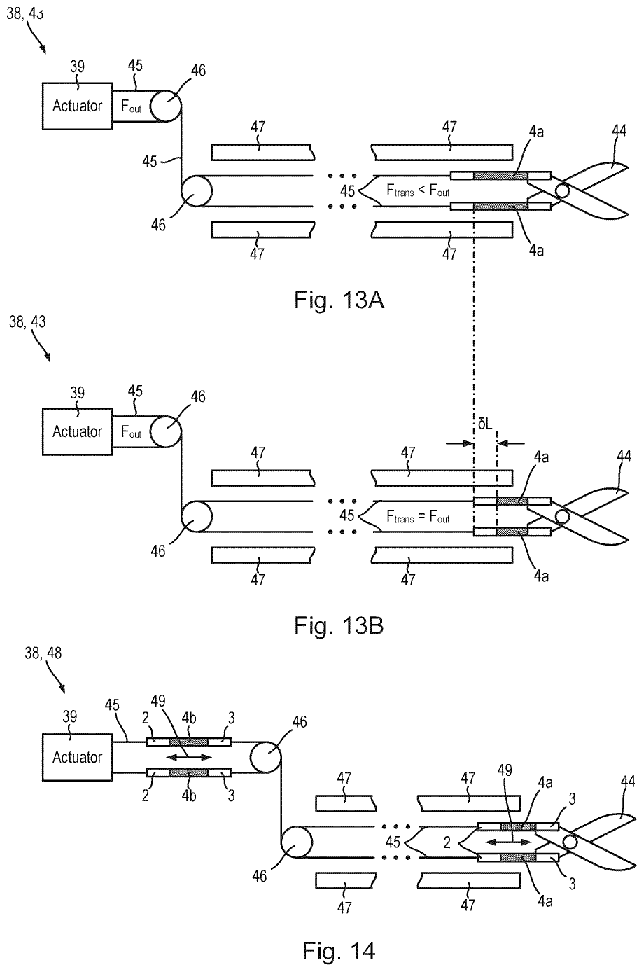

Referring also to FIGS. 13A and 13B, the force correction method shall be illustrated with reference to the first tool 43.

Referring in particular to FIG. 13A, the first SMA elements 4a are used to measure the transmitted force Fₜᵣₐₙₛ and it is found to be less than the actuator 39 output force Fₒᵤₜ, Fₜᵣₐₙₛ<Fₒᵤₜ. The difference ΔF=Fₒᵤₜ−Fₜᵣₐₙₛ manifests as a reduced tension in the cables 45 at the distal (gripper 44) end, and arises from a variety of sources including, but not limited to, friction between the cables 45 and cable sheathe 47, mechanical compliance of the cables 45 and/or pulleys 46, and so forth.

Referring in particular to FIG. 13B, the first SMA elements 4a are actuated to reduce their length by an amount δL, increasing the tension at the distal end to compensate for losses until the transmitted force Fₜᵣₐₙₛ is equal to the output force Fₒᵤₜ. In this way, the actuation of the first SMA elements 4a may remove slack caused by the cable-pulley system.

Although measurement of the transmitted force Fₜᵣₐₙₛ has been described using the first SMA element(s) 4a for both measurement and actuation, in other examples the first SMA elements 4a may be used purely for actuation, and the transmitted force Fₜᵣₐₙₛ may instead by measured using a force sensor (of any suitable type) separate from the first SMA element(s) 4a.

### Fluctuation/Jitter Corrections—Stabilised Mode

Additionally and/or alternatively to implementing force corrections to match an average output force Fₒᵤₜ, the tool 38 may also implement force corrections to compensate for fluctuations in the output force Fₒᵤₜ.

For example, such fluctuations may be a result of handshake when the actuator 39 is manual, such as during surgical operations using a manual actuator 39 tools. Even if the actuator 39 is not manual, there may be fluctuations, for example generated in the actuator 39 itself and/or propagated from fluctuations in an electrical/hydraulic/pneumatic supply.

The first SMA element 4a may be used as an actuator to actively stabilise the motion of the tool 38 (specifically the operating element 40). Such a “stabilised mode” may be active all the time, or it may be selectable by a user when desired. The transmitted force Fₜᵣₐₙₛ is measured and compared to a target force F_{targ} to determine a force difference ΔF_{fluc}=F_{targ}−Fₜᵣₐₙₛ. In response to detecting a non-zero difference ΔF_{fluc}, the controller 6 actuates the first SMA element 4a (heating or cooling depending on sign of the difference ΔF_{fluc}) to compensate and remove the difference ΔF_{fluc}.

The target force F_{targ} may be predetermined, user determined or automatically determined. As an example of automatic determination, the target force F_{targ} may be determined based on a moving window average of recently measured force values buffered in the controller 6. In this way, the fluctuation correction may act as a mechanical low-pass filter, allowing slow, controlled adjustments whilst smoothing out rapid changes. The size of the moving window may be adjustable to allow tuning of such effects, even in use.

As an example of user determined target force F_{targ}, a user may adjust the tool 38 to a desired force, then actuate a switch or toggle element provided on the tool 38 to activate the stabilised mode and lock the transmitted force Fₜᵣₐₙₛ to the target force F_{targ}, which in this instance could be the transmitted force Fₜᵣₐₙₛ at the time of activating stabilisation (but preferably an average of the transmitted force Fₜᵣₐₙₛ over a period for accuracy). This may be particularly advantageous when the actuator 39 is manually driven, when it is desired to maintain a precise force for a period of time.

Returning to the example of the cable-pulley driven first tool 43, when the actuator 39 is manually driven, unwanted motions of a surgeon's hand may be removed using the stabilised mode.

When the actuator 39 is not manually driven, the target force F_{targ} may correspond to the intended actuator output force Fₒᵤₜ. This may allow combining the force corrections for both mechanical losses in the force transmission mechanism 41 and fluctuations. For example, in order to set an intended target force F_{targ}, the tool may look-up a corresponding drive current for a motor etc.

Although measurement of the transmitted force Fₜᵣₐₙₛ has been described using the first SMA element(s) 4a for both measurements and actuation, in other examples the first SMA elements 4a may be used purely for actuation, and the transmitted force Fₜᵣₐₙₛ may instead by measured using a force sensor (of any suitable type) separate from the first SMA element(s) 4a.

### Friction Reduction

Mechanical components have to overcome friction to move relative to one another, and initial (static) frictional forces tend to drop to lower values once movement begins (dynamic friction). SMA elements 4a, 4b may be used to maintain a force transmission mechanism 41 in motion to avoid the “stickiness” that can be associated with overcoming static frictional forces.

For this approach, the force transmission mechanism 41 of the tool 38 includes the second SMA element 4b. The second SMA element 4b is separated from the first SMA element 4a by one or more linkages 42 of the force transmission mechanism 41, is connected in series with the first SMA element 4a. Preferably the second SMA element 4b is the ultimate or penultimate element of the force transmission mechanism 41 moving from the operating element 40 to the actuator 39.

The controller 6 is then configured to cyclically heat the first SMA element 4a and the second SMA element 4b to cause vibrations of the intervening linkages 42 of the force transmission mechanism 41. The generated vibrations of the force transmission mechanism 41 are preferably of low amplitude and a frequency of greater than or equal to 10 Hz. Preferably the vibrations are at a frequency greater than or equal to 20 Hz, more preferably greater than or equal to 50 Hz. The vibrations should preferably not alter the force F being transmitted between the first SMA element 4a and the second SMA element 4b (excepting intentional corrections as described hereinbefore). In other words, contracting the second SMA element 4b is matched by allowing the first SMA element to expand and vice versa. Closed loop control is preferably implemented to ensure that any contraction of the first SMA element 4a is equal to the extension of the other, opposing second SMA element 4b, and vice versa.

The generated vibrations of the force transmission mechanism 41 may help to reduce sticking in the force transmission mechanism 41 arising from one or more elements transitioning between static and dynamic friction.

In an tool 38 which includes two (or more) parallel SMA elements 4 providing actuation, for example for steering an operating element 40, the phase of vibrations generated may be considered. For example, vibrations in two (or more) parallel SMA elements 4 could be configured such that a phase between the vibrations of the two parallel actuators minimises any motion of the operating element (at the frequency of vibration).

Referring also to FIG. 14, a second exemplary surgical tool 48 (hereinafter the “second tool”) is shown.

The second tool 48 is the same as the first tool 43, except that it further includes a pair of second SMA elements 4b coupling the input to the cable pulley system to the actuator 39. The second SMA elements 4b may be used for force measurements, for example to measure output forces Fₒᵤₜ for input to correction/stabilisation methods described hereinbefore.

Furthermore, the first 4a and second 4b SMA elements may be actuated together to generate vibrations 49 with the aim of reducing friction in the cable pulley system.

The above-described force sensors and tools comprise at least one SMA element. The term ‘shape memory alloy (SMA) element’ may refer to any element comprising SMA. The SMA element may be described as an SMA wire. The SMA element may have any shape that is suitable for the purposes described herein. The SMA element may be elongate and may have a round cross section or any other shape cross section. The cross section may vary along the length of the SMA element. The SMA element might have a relatively complex shape such as a helical spring. It is also possible that the length of the SMA element (however defined) may be similar to one or more of its other dimensions. The SMA element may be sheet-like, and such a sheet may be planar or non-planar. The SMA element may be pliant or, in other words, flexible. In some examples, when connected in a straight line between two components, the SMA element can apply only a tensile force which urges the two components together. In other examples, the SMA element may be bent around a component and can apply a force to the component as the SMA element tends to straighten under tension. The SMA element may be beam-like or rigid and may be able to apply different (e.g. non-tensile) forces to elements. The SMA element may or may not include material(s) and/or component(s) that are not SMA. For example, the SMA element may comprise a core of SMA and a coating of non-SMA material. Unless the context requires otherwise, the term ‘SMA element’ may refer to any configuration of SMA material acting as a single actuating element which, for example, can be individually controlled to produce a force on an element. For example, the SMA element may comprise two or more portions of SMA material that are arranged mechanically in parallel and/or in series. In some arrangements, the SMA element may be part of a larger SMA element. Such a larger SMA element might comprise two or more parts that are individually controllable, thereby forming two or more SMA elements. The SMA element may comprise an SMA wire, SMA foil, SMA film or any other configuration of SMA material. The SMA element may be manufactured using any suitable method, for example by a method involving drawing, rolling or deposition and/or other forming process(es). The SMA element may exhibit any shape memory effect, e.g. a thermal shape memory effect or a magnetic shape memory effect, and may be controlled in any suitable way, e.g. by Joule heating, another heating technique or by applying a magnetic field.

The force sensors 1 described herein may be, or may be provided in, any one of the following devices: a smartphone, a protective cover or case for a smartphone, a functional cover or case for a smartphone or electronic device, a camera, a foldable smartphone, a foldable smartphone camera, a foldable consumer electronics device, a camera with folded optics, an image capture device, an array camera, a 3D sensing device or system, a servomotor, a consumer electronic device, a mobile or portable computing device, a mobile or portable electronic device, a laptop, a tablet computing device, an e-reader, a computing accessory or computing peripheral device, an audio device, a security system, a gaming system, a gaming accessory, a robot or robotics device, a medical device, an augmented reality system, an augmented reality device, a virtual reality system, a virtual reality device, a wearable device, a drone, an aircraft, a spacecraft, a submersible vessel, a vehicle, and an autonomous vehicle, a tool, a surgical tool, a remote controller, clothing, a switch, dial or button, a display screen, a touchscreen, a flexible surface, a wireless communication device, a tool, a surgical tool, and a manufacturing machine. It will be understood that this is a non-exhaustive list of example devices. Tools 38 described herein may take the form of surgical tools, robotic surgical tools, and tools used for manufacturing. It will be understood that this is a non-exhaustive list of example applications.

1. A method of determining a force applied between a first part and a second part connected to the first part via a shape memory alloy, SMA, element such that force is transferred via the SMA element, wherein the force is determined based on a resistance of the SMA element or an amount of energy applied to the SMA element in order to maintain a resistance of the SMA element at a predetermined value.

2. The method according to claim 1, wherein a majority of the force applied between the first part and the second part is transferred via the SMA element.

3. The method according to claim 1, wherein the force is applied along the SMA element parallel to a first direction, and wherein the SMA element is configured such that the applied force corresponding to the upper plateau stress of the material of the SMA element varies with position along the first direction.

4. The method according to claim 1, wherein the SMA element is configured such that the applied force generates a stress gradient along the SMA element.

5. method according to claim 1, wherein a temperature gradient is applied or generated along the SMA element.

6. The method according to claim 1, wherein a cross-sectional area of the SMA element tapers along the length of the SMA element.

7. The method according to claim 1, wherein at least part of the length of the SMA element is embedded in, or bonded to, a second material, and wherein the spring constant of the second material tapers along the length of the SMA element; or
wherein the SMA element is directly connected to the first part, and a portion of the length of the SMA element is engaged with a block of compliant material which couples the SMA element to the second part.

8. (canceled)

9. The method according to claim 1, wherein determining the force based on an amount of energy applied to the SMA element in order to maintain a resistance of the SMA element at a predetermined value comprises:
controlling a power applied to cause resistive heating of the SMA element in order to maintain the length of the SMA element at a constant value; and
determining the applied force based on the power applied.

10. The method according to claim 9, further comprising an initial step of controlling a power applied to cause resistive heating of the SMA element such that a power history enables determining the position of the SMA element in a hysteresis behaviour of the SMA material.

11. A force sensor comprising a first part and a second part connected to the first part via a shape memory alloy, SMA, element such that in response to a force applied between the first and second parts, a force is transferred via the SMA element;
wherein the force sensor is configured to determine the force applied between the first and second parts based on a resistance of the SMA element or an amount of energy applied to the SMA element in order to maintain a resistance of the SMA element at a predetermined value.

12. The force sensor according to claim 11, wherein the force is applied along the SMA element parallel to a first direction, and wherein the SMA element is configured such that the minimum applied force necessary for superelastic deformation of the SMA element varies with position along the first direction.

13. The force sensor according to claim 11, wherein the SMA element is configured such that the applied force generates a stress gradient along the SMA element.

14. The force sensor according to claim 11, configured to apply or generate a temperature gradient along the SMA element.

15. The force sensor according to claim 11, wherein a cross-sectional area of the SMA element tapers along the length of the SMA element.

16. The force sensor according to claim 11, wherein at least part of the length of the SMA element is embedded in, or bonded to, a second material, and wherein the spring constant of the second material tapers along the length of the SMA element; or
wherein the SMA element is directly connected to the first part, and wherein a portion of the length of the SMA element is engaged with a block of compliant material which couples the SMA element to the second part.

17. (canceled)

18. A controller configured to determine a force applied between a first part and a second part of a force sensor according to claim 11, wherein the force is determined based on a resistance of the SMA element or an amount of energy applied to the SMA element in order to maintain a resistance of the SMA element at a predetermined value.

19. The controller according to claim 18, further configured to supply power to cause resistive heating of the SMA element.

20. The controller according to claim 19, further configured to:
control the power applied to cause resistive heating of the SMA element in order to maintain the length of the SMA element at a constant value; and
determine the applied force based on the power applied.

21. A computer program stored on a non-transitory computer readable medium, wherein when executed by a digital electronic processor, the computer program causes the digital electronic processor to carry out the method according to claim 1.

22. Apparatus comprising the force sensor according to claim 11, and the controller according to claim 18 connected to the force sensor.

23.-39. (canceled)
